(19) United States
(12) Patent Application Publication
(10) Pub. No.: US 2024/0350414 A1
(43) Pub. Date: Oct. 24, 2024

(54) DIRECT TABLETING AUXILIARY COMPOSITION

(71) Applicant: BASF SE, Ludwigshafen (DE)

(72) Inventors: Silke Gebert, Ludwigshafen (DE); Felicitas Guth, Ludwigshafen (DE); Thorsten Cech, Ludwigshafen (DE); Karl Kolter, Limburgerhof (DE)

(21) Appl. No.: 18/684,728

(22) PCT Filed: Aug. 19, 2022
(86) PCT No.: PCT/EP2022/073172

§ 371 Date: Feb. 19, 2024

(30) Foreign Application Priority Data
Aug. 25, 2021 (EP) 21192938.5

(51) Int. Cl. A61K 9/20; A61K 31/192; A61K 31/451

(52) U.S. Cl. CPC A61K 9/2031; A61K 9/2013; A61K 9/2018; A61K 9/2027; A61K 31/192; A61K 31/451

(57) ABSTRACT

The present invention relates to a direct tableting auxiliary composition based on lactose powder mixed intimately with a water-soluble polyethylene glycol-polyvinyl alcohol graft copolymeras binder, crosslinked insoluble polyvinylpyrrolidone as tablet disintegrating agent and a lubricant.

The present invention relates to a direct tableting auxiliary composition based on lactose powder mixed intimately with a water-soluble polyethylene glycol-polyvinyl alcohol graft copolymer as binder, crosslinked insoluble polyvinylpyrrolidone as tablet disintegrating agent, preferably Kollidon CL-F and a lubricant.

In the pharmaceutical industry the most commonly employed means to deliver APIs (active pharmaceutical ingredients) is the tablet, which may be obtained through the compression of appropriately formulated powders. Conventional, compressible mixtures are typically obtained by the blending of an API and suitable excipient materials. These excipients may include diluents or fillers, binders, or adhesives, disintegrants, glidants or flowability agents, colours, flavors and mixtures thereof.

As mentioned in WO2008/020990 these materials may simply be blended or may be granulated in either the dry or wet state. Once mixing is completed, a lubricant is added and the material compressed into tablets. WO2008/020990 itself is striving for a universal excipients blend that maximises the ability of APIs to be formulated without adversely affecting its safety and efficacy profiles. The blend would be mixed with an API and optionally a lubricant, then compressed into tablets. This is also confirmed in U.S. Pat. No. 3,344,030. The flow charts at page 14, 15 and 17-19 of WO2007/031933 perfectly demonstrate that a lubricant should be added after granulation and milling of the granule, only to be followed by compaction. WO2001/41744, at page 31 in particular, teaches to prepare pellets by coating spheroids containing an API and pre-mixed excipient components with a suspension containing an API and pre-mixed excipients with a suspension containing magnesium stearate. Independent on the way of tableting a lubricant is blended with filler and active compound only just prior to compression.

Likewise, U.S. Pat. No. 5,006,345, provides a direct tableting auxiliary based on lactose powder mixed intimately with a binder, and a tablet disintegrant agent. After mixing these with the API, a lubricant is only then added and the mixture is pressed to produce tablets. WO97/44014, US2006/0246135, WO2007/086689, U.S. Pat. No. 6,514,524 and WO2002/03963 teach similarly. In all events, a lubricant is again added immediately preceding compaction, after pre-mixing of the other excipient components. Outside the field of providing ready-to-use co-processed tablet excipient components, WO2004/110406 and US2006/0247234 both disclose processes in which the API, excipients and lubricant are mixed altogether.

In the field, however, a lubricant is deemed necessary to realise release of the compressed form or tablet from the device. However, at the same time it is believed that the lubricant could affect the necessary binding between the various carrier components, and, in the case of hydrophobic lubricants such as magnesium stearate, tablet disintegration properties negatively. Regarding the binding properties, the reasons would rest in the tendency of lubricants to coat the excipient components, thus preventing these from adhering from to one another. Also, a hydrophobic lubricant coat repels water which plays an important role in disintegration. Therefore, the use of lubricant is postponed until mixing of all components has been achieved, so as to minimize contact time between lubricant and other tablet components prior to the actual compression step.

In the art, WO2009/112287 disclosed that the lubricant can already be added to the coprocessing step, thus providing a ready-to-use excipient composition which would conveniently require only the steps of adding the APIs and compression, and still expedite ejection from the tableting die. Despite the use of lubricant at an earlier processing stage, the die ejection force and tablet force are excellent. However, to yield these properties, the process of WO2009/112287 involves spraying of the lubricant onto the cogranulated components, thus creating a lubricant coat covering the granules. It thus continues to feed the skilled persons belief that the lubricant should make direct contact with the die, which can only be achieved when spraying it as an outside layer to the excipient composition.

WO2011/074961 deals with co-processed excipient compositions comprising granules, said granules comprising at least one filler-binder and at least one lubricant which have been subjected to granulation together, said granules being coated with lactose. It was found that flowability can be increased without sacrificing disintegration. The developed excipient is said to have overcome the obligatory final coating of excipients with lubricants which complicates tablet manufacture and loss of compactability.

In the art, there is however the continuous need for new excipient compositions and for further optimizing the process for producing ready-to-use co-processed excipient compositions. Especially there remains the need in the industry for continuous manufacturing concepts. Continuous manufacturing concepts for pharmaceutical excipients which exhibit a good flowability while simultaneously enabling acceptable ejection forces during tableting and having a high hardness and fast disintegration time of the compressed dosage form. Such a performance is requested for a variety of drug containing tablets e.g. analgesics.

Thus in one aspect the present invention provides a direct tableting auxiliary composition comprising at least one lactose component, at least one water soluble polyethylene glycol-polyvinyl alcohol graft copolymer, at least one crosslinked insoluble polyvinylpyrrolidone and at least lubricant.

Lactose belongs to the group of disaccharides and consists of the two molecules β-D-galactose and α/β-D-glucose, which are linked together by a β-1,4 glycosidic bond. According to the invention, the lactose component may be an anhydrous lactose or a lactose monohydrate. Lactose monohydrate is preferred, since it is less hygroscopic compared to anhydrous lactose and is thus more suitable in compositions containing water-sensitive pharmaceutically active ingredients. More preferred is a lactose monohydrate having a content of amorphous lactose monohydrate of less than 5% by weight.

Polyethylene glycol-polyvinyl alcohol graft copolymer is a white to yellowish powder that dissolves easily in water with a low viscosity and has the advantage not to be oxygen sensitive and not to build hydrogen peroxides like other polymers do. It is a graft polymer, comprising polyethylene glycol and polyvinyl alcohol, bound in a ratio of 25:75. It has a molecular weight of approximately 45000. The addition of polyethylene glycol-polyvinyl alcohol graft copolymer enables both the particle size distribution to be optimized and unexpectedly, to keep the disintegration time of the tablets low despite the use of a water-soluble binder. The powder of polyethylene glycol-polyvinyl alcohol graft copolymer is marketed as Kollicoat®IR and contains approximately 0.3% colloidal silica. The polyethylene glycol and polyvinyl alcohol graft copolymer used in this invention does not contain any colloidal silica. The direct tableting auxiliary composition does remarkably contain no glidant/flowability agent at all, and still exhibits extraordinary high flowability.

The crosslinked insoluble polyvinylpyrrolidones are widely used in the pharmaceutical industry because of their swelling properties. They are thus predominantly used as disintegrants in tablets. Furthermore their application as pharmaceutical excipient is triggered by their ability to hydrophilize insoluble drugs, to stabilize suspensions and to form complexes, as well as their adsorptive properties. According to the invention the crosslinked polyvinylpyrrolidone may be Kollidon CL-SF and/or Kollidon CL-F, whereby Kollidon CL-F is preferred.

The direct tableting auxiliary composition according to the invention preferably comprises the lactose component in an amount of 75-98% by weight, more preferably 80-95% by weight and even more preferably 83-92% by weight based on the total mass of the composition.

The crosslinked insoluble polyvinylpyrrolidone component is preferably present in an amount of 1-15%-by weight, more preferably 2-12% by weight and even more preferably 3-10% by weight based on the total mass of the composition.

The polyethylene glycol-polyvinyl alcohol graft copolymer may be comprised in the composition in an amount of 0.5-10% by weight, more preferably 1-10% by weight and even more preferably 2-9% by weight based on the total mass of the composition.

The composition of the invention preferably has a total amount of lubricant of 0.5-10% by weight more preferably 1-10% by weight and even more preferably 2-9% by weight based on the total mass of the composition, wherein the sum of all components of the composition adds up to 100% by weight. According to the invention the lubricant is sodium stearyl fumarate, magnesium stearate, stearic acid and/or poloxamer 407 (Kolliphor P 407 micro), preferably it is sodium stearyl fumarate, and magnesium stearate and most preferably it is sodium stearyl fumarate.

The direct tableting auxiliary composition has a spherical morphology and is preferably present in the form of granules, whereby the mean particle size (d50) of said granules is preferably in the range of from 50-500 μm, more preferably of from 80-300 μm and even more preferably in the range of from 100-250 μm.

During the tableting process, the composition of the invention may be mixed with at least one API with API levels ranging from 1 to 75% by weight.

In another aspect the composition, preferably the granule is free of any API.

In a further aspect of the invention, a method for manufacturing a composition as described above is provided. The method of the invention comprises the steps of (i) providing a solution or suspension comprising at least one polyethylene glycol-polyvinyl alcohol graft copolymer, at least a crosslinked insoluble polyvinylpyrrolidone and/or at least one lactose in a liquid medium, and (ii) spraying the solution or suspension obtained in step (i) in an environment at an increased temperature, optionally at reduced pressure, thereby removing the liquid medium. In step (i), the at least one polyethylene glycol-polyvinyl alcohol graft copolymer component, the at least one crosslinked insoluble polyvinylpyrrolidone and the at least one lactose component are preferably at least partially solved in a liquid medium, such as water, or an organic solvent, such as ethanol, acetic acid and acetone, and mixtures thereof.

In step (ii), the solution or suspension obtained in step (i) is sprayed whereby in contrast to the state of the art the lubricant is not applied as solution or suspension but as a solid powder to the surface of the spray dried particles.

It is particularly advantageous that this new process could be integrated into a continuous manufacturing process without additional preparation steps.

In another aspect the present invention relates to a composition as obtained by the above described process. It has turned out that this process results in composition with excellent flowability, a high bulk density and excellent tableting properties. In a further aspect the present invention is directed to the use of the composition as described above as an excipient in making oral dosage forms, particularly as a tableting excipient, more particularly as a direct tableting excipient. Due to the excellent flowability and high bulk density, the composition is also very suitable as diluent for binary mixtures with APIs to be filled into hard shell capsules (e.g. hard gelatin capsules).

It has turned out that the use of the composition according to the invention as a direct tableting excipient in standard tablet formulations results in in a significant improvement in tablet hardness combined with a very low disintegration time of the tablets.

The invention is further illustrated by the following FIGURE and examples.

### FIGURES

FIG. 1: SEM Image of granules according to the invention

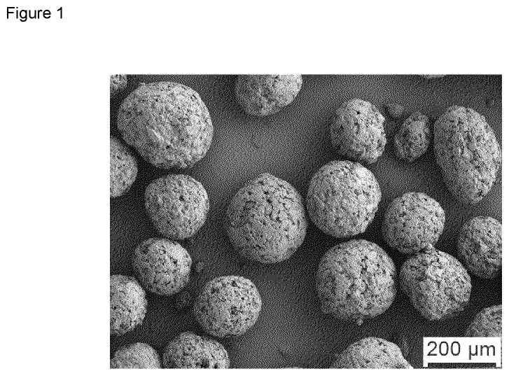

### EXAMPLES

### Methods

The tamped density of the direct tableting auxiliary composition was measured in accordance with Chapter 2.9.34 method 2 of the European Pharmacopeia 9.

The bulk density of the direct tableting auxiliary composition was measured in accordance with Chapter 2.9.34 method 3 of the European Pharmacopeia 9.

Hausner ratio equals the quotient of tamped density and bulk density.

The packing fraction equals the quotient of bulk density and true density

Flowability and angle of repose of the direct tableting auxiliary composition are determined in accordance with Chapter 2.9.16 and Chapter 2.9.36 of the European Pharmacopeia 9.

Particle size distribution (D10, D50, D90) of the direct tableting auxiliary composition were determined using a Malvern Mastersizer 2000.

Tablet disintegration was measured in accordance with Chapter 2.9.1 test A of the European Pharmacopeia 9

Tablet hardness was measured in accordance with Chapter 2.9.8 of the European Pharmacopeia 9 using a Sotax HT 100 tablet tester, the tablet hardness being determined successively on 20 tablets with a speed of the test jaw of 120 mm/min.

True density was measured at 23° C., according to EN ISO 1183-3 (gas pycnometer). Gas Pyknometer: Micromeritics, AccuPyc 1340; volume metering chamber 10 cm3; calibration with steel balls. Prior to the measurement the samples were dried overnight in a vacuum oven (Fa. Heraeus) at 23° C. and 5 hPa.

### Example 1: Manufacturing of the Direct Tableting Auxiliary Composition According to the Invention

Crosslinked insoluble polyvinylpyrrolidone (e.g. Kollidon CL-F) and a solution of Kollicoat® IR (not containing any colloidal silica) in water were suspended in water and the suspension cooled down to less than 20° C. Under stirring lactose (e.g. GranuLac) was continuously dosed to the suspension. For removing the solvent the obtained suspension was sprayed dried at an inlet air temperature of 155° C.±5° C., outlet air temperature of >80° C., whereby the sodium stearyl fumarate (e.g. PRUV or Alubra) was dosed into the spray dryer in dry form and afterwards cooled down, whereby the fines were separated from the granules by a cyclone. The direct tableting auxiliary had the composition, shown in Table 1.

TABLE 1

| | | Amount in |
|---|---|---|
| 1 | Ingredient | % by weight |
| | Lactose | 86.5 |
| | polyethylene glycol-polyvinyl | 3.5 |
| | alcohol graft copolymer | |
| | crosslinked insoluble polyvinylpyrrolidone | 9 |
| | sodium stearyl fumarate | 1 |

Further compositions of the auxiliary were made using the conditions described above replacing the binder polyethylene glycol-polyvinyl alcohol graft copolymer by using a different binder and by changing the amounts, reducing or enhancing the lactose amount respectively (Table 2).

TABLE 2

| | | | | | Angle of | Bulk | |
|---|---|---|---|---|---|---|---|
| | | D10 | D50 | D90 | repose | density | Disintegration |
| | Binder | [μm] | [μm] | [μm] | [°] | (g/ml) | (s) |
| 1 | polyethylene | 72 | 141 | 251 | 27 | 0.560 | 187 |
| | glycol-polyvinyl | | | | | | |
| | alcohol graft copolymer | | | | | | |
| | (3.5% by weight) | | | | | | |
| 2 | polyethylene | 33 | 98 | 190 | 31 | 0.518 | 112 |
| | glycol-polyvinyl | | | | | | |
| | alcohol graft | | | | | | |
| | copolymer | | | | | | |
| | (0% by weight) | | | | | | |
| 3 | polyethylene | 96 | 195 | 338 | 26 | 0.495 | 239 |
| | glycol-polyvinyl | | | | | | |
| | alcohol graft | | | | | | |
| | copolymer (6.5% by | | | | | | |
| | weight) | | | | | | |
| 4 | Kollidon VA64 | 67 | 137 | 240 | 27 | 0.543 | 411 |
| | (3.5% by weight) | | | | | | |
| 5 | Kollidon 90 (3.5% | 69 | 161 | 282 | 26 | 0.540 | >900 |
| | by weight) | | | | | | |

TABLE 3
The Hausners Ratio for the compositions of Table 2

| | Binder | Hausner Ratio |
|---|---|---|
| 1 | polyethylene glycol-polyvinyl alcohol | 1.21 |
| | graft copolymer (3.5% by weight) | |
| 2 | polyethylene glycol-polyvinyl alcohol | 1.23 |
| | graft copolymer (0% by weight)r | |
| 3 | polyethylene glycol-polyvinyl alcohol | 1.05 |
| | graft copolymer (6.5% by weight) | |
| 4 | Kollidon VA64 (3.5% by weight) | 1.18 |
| 5 | Kollidon 90 (3.5% by weight) | 1.11 |

Further compositions of the auxillary were made using the conditions described above replacing the disintegrant Kollidon CL-F by a different disintegrant Kolldon CL-SF and changing the amounts, by reducing or enhancing the lactose amount respectively.

TABLE 4

| | | Disintegration |
|---|---|---|
| | Disintegrant | time [s] |
| 1 | Kollidon CL-F (3.5% by weight) | 199 |
| 6 | Kollidon CL-F (0% by weight) | 313 |
| 7 | Kollidon CL-F (10% by weight) | 136 |
| 8 | Kollidon CL-SF (3.5% by weight) | 171 |
| 9 | Kollidon CL-SF (10% by weight) | 121 |

TABLE 5
Lubricant is dosed into the spray dryer in dry form (external).
Lubricant is added as suspension into the disintegrant
suspension, then mixed and spray dried (internal).

| | lubricant | [N] |
|---|---|---|
| | external | |
| 10 | sodium stearyl fumarate (0% by weight) | >1000 |
| 11 | sodium stearyl fumarate (0.5% by weight) | 192 |
| 12 | sodium stearyl fumarate (1% by weight) | 180 |
| 13 | sodium stearyl fumarate (4% by weight) | 167 |
| 14 | Mg-stearate (0.5% by weight) | 181 |
| 15 | Mg-stearate (4% by weight) | 152 |
| 16 | Stearic acid (0.5% by weight) | 320 |
| 17 | Stearic acid (4% by weight) | 198 |
| | internal | |
| 18 | sodium stearyl fumarate (1% by weight) | 247 |
| 19 | sodium stearyl fumarate (2% by weight) | 259 |
| 20 | sodium stearyl fumarate (3% by weight) | 272 |

TABLE 6
Competitor product comparison against
inventive auxiliary composition

| | | | | Angle | | | |
|---|---|---|---|---|---|---|---|
| | | | | of | Bulk | | |
| | D10 | D50 | D90 | repose | density | Hausner | Packing |
| Binder | [μm] | [μm] | [μm] | [°] | (g/ml) | Ratio | fraction |
| Ludipress | 66 | 214 | 474 | 33 | 0.545 | 1.07 | 0.36 |
| Cellactose | 40 | 131 | 261 | 35 | 0.421 | 1.22 | 0.27 |
| 80 | | | | | | | |
| CombiLac | 38 | 145 | 287 | 28 | 0.483 | 1.17 | 0.31 |
| MicoceLac | 42 | 139 | 268 | 29 | 0.488 | 1.18 | 0.32 |
| 100 | | | | | | | |
| StarLac | 25 | 113 | 244 | 28 | 0.588 | 1.19 | 0.38 |
| Prosolv | 39 | 120 | 244 | 36 | 0.410 | 1.20 | 0.26 |
| Easytab | | | | | | | |
| SP | | | | | | | |
| Inventive | 74 | 156 | 274 | 26 | 0.540 | 1.11 | 0.35 |
| auxiliary | | | | | | | |

### Example 2: Tableting of the Direct Tableting Auxiliary Composition According to the Invention

TABLE 7

| | A | B |
|---|---|---|
| Formulation 1 (example 1) | 60 | |
| Prosolv Easytab SP | | 60 |
| Ibuprofen | 40 | 40 |

TABLE 8

| | | | | Angle of repose (°) of |
|---|---|---|---|---|
| | Compression | Hardness | Disintegration | the composition/ |
| | force (kN) | (N) | time (s) | ibuprofen blend |
| A | 6.3 | 60 | 65 | 37 |
| | 9.4 | 94 | 130 | |
| | 14.1 | 121 | 131 | |
| | 18.8 | 142 | 179 | |
| | 23.6 | 149 | 257 | |
| B | 6.3 | 48 | 48 | 53 |
| | 9.4 | 90 | 127 | |
| | 14.1 | 113 | 139 | |
| | 18.8 | 129 | 191 | |
| | 23.6 | 137 | 304 | |

### Tabletting

Compaction simulator STYL′One EVO
Punch: 10 mm, flat
Compression forces: 6.3, 9.4, 14.1, 18.8, 23.6 kN

TABLE 9

| | A |
|---|---|
| Formulation 1 (example 1) | 98.7 |
| Loperamide HCl | 1.3 |

TABLE 10

| | Compression | Hardness | Disintegration | Content |
|---|---|---|---|---|
| | force (kN) | (N) | time (s) | uniformity (%) |
| A | 2 | 16 | 23 | 98 |
| | 4 | 45 | 35 | 97 |
| | 6 | 76 | 53 | 101 |
| | 8 | 104 | 71 | 98 |
| | 10 | 123 | 89 | 95 |
| | 12 | 141 | 118 | 102 |

### Tabletting

Compaction simulator STYL′One EVO
Punch: 7 mm, flat
Compression forces: 2, 4, 6, 8, 10, 12 kN

1. A direct tableting auxiliary composition comprising
A) 75-98% by weight of a lactose suitable for tableting,
B) 0.5-10% by weight of a water-soluble polyethylene glycol-polyvinyl alcohol graft copolymer,
C) 1-15% by weight of a crosslinked insoluble polyvinylpyrrolidone,
D) 0.5-10% by weight of a lubricant,
in a form of spherical granules,
and wherein a sum of all components A to D adds up to 100% by weight and a mean particle size (D50) of said spherical granules is in a range of from 50 μm to 500 μm.

2. The direct tableting auxiliary composition according to claim 1 with a mean particle size of 80 μm to 300 μm.

3. The direct tableting auxiliary composition according to claim 1 with a mean particle size of 100 μm to 250 μm.

4. The direct tableting auxiliary composition according to claim 1, wherein the lactose is lactose monohydrate.

5. The direct tableting auxiliary composition according to claim 4 wherein the lactose monohydrate has a content of amorphous lactose monohydrate of less than 5% by weight.

6. The direct tableting auxiliary composition according to claim 1, wherein the insoluble polyvinylpyrrolidone is Kollidon CL-SF and/or Kollidon CL-F.

7. The direct tableting auxiliary composition according to claim 6, wherein the insoluble polyvinylpyrrolidone is Kollidon CL-F.

8. The direct tableting auxiliary compositions according to claim 1, wherein the lubricant is sodium stearyl fumarate, magnesium stearate, and/or stearic acid poloxamer 407 (Kolliphor P 407 micro).

9. The direct tableting auxiliary composition according to claim 8, wherein the lubricant is magnesium stearate.

10. The direct tableting auxiliary composition according to claim 8, wherein the lubricant is stearic acid.

11. The direct tableting auxiliary composition according to claim 8, wherein the lubricant is sodium stearyl fumarate.

12. The direct tableting auxiliary composition according to claim 1, wherein the lubricant is located on a surface of said granules.

13. The direct tableting auxiliary composition according to claim 1 having a Hausner Ratio of from 1.1 to 1.4.

14. The direct tableting auxiliary composition according to claim 1 having a packing fraction of from 0.31 to 0.38.

15. The direct tableting auxiliary composition according to claim 1 having an angle of repose of from 25° to 31°.

16. A continuous process for preparing the direct tableting auxiliary composition of claim 1, comprising spray drying the lactose, polyethylene glycol-polyvinyl alcohol graft copolymer, crosslinked insoluble polyvinylpyrrolidone suspension, whereby the lubricant is dosed in dry powder form.

17. A process according to claim 16 where the lubricant is added as dry powder that leads to a product where the lubricant adheres to the surface of the spray dried particles.

18. A cosmetic or pharmaceutical preparation, a preparation of agrochemical actives, a preparation in the sector of food, feed and food or feed supplementation comprising a direct tableting auxiliary composition of claim 1.
